(19) United States
(12) Patent Application Publication
(10) Pub. No.: US 2022/0265947 A1
(43) Pub. Date: Aug. 25, 2022

(54) ENDOTRACHEAL TUBE FASTENING APPARATUS AND USE THEREOF

(71) Applicant: Joaquim Da Cunha Campos Goncalves, Cuiabá (BR)

(72) Inventor: Joaquim Da Cunha Campos Goncalves, Cuiabá (BR)

(21) Appl. No.: 17/528,916

(22) Filed: Nov. 17, 2021

Related U.S. Application Data
(63) Continuation of application No. 17/181,646, filed on Feb. 22, 2021, now abandoned.

(51) Int. Cl. A61M 16/04; A61M 16/00

(52) U.S. Cl. CPC A61M 16/049; A61M 16/0003

(57) ABSTRACT

An endotracheal tube fastening apparatus and use thereof for the field of medical apparatuses, specifically to the field of useful apparatuses for fastening devices related to advanced life support to a patient. The present invention discloses an endotracheal tube adhesive fastening apparatus, specifically to endotracheal tubes used for patients' intubation during surgical procedures whenever an artificial respiration system is required. The apparatus of the present invention has a shape that allows complete isolation of the oral cavity during the procedure, as well as ensuring tube stability during its fastening and removal, in order to reduce the possibility of injuries to the patient's airways or hypoxia due to accidental extubation.

### REFERENCE TO RELATED APPLICATIONS

The present application is a continuation of and claims priority to U.S. patent application Ser. No. 17/181,646, filed on Feb. 22, 2021, which is hereby incorporated by reference herein in its entirety.

### FIELD OF APPLICATION

The present invention refers to the field of medical apparatuses, more precisely to the field of apparatuses useful for fastening devices that influence the respiratory system of patients and allow mechanical ventilation to be instituted.

Specifically, the present invention discloses and explains an apparatus useful in fastening endotracheal tubes, specifically orotracheal tubes, used during surgical procedures requiring advanced life support to be provided to patients, for example, procedures done under general anesthesia.

Intubation ensures the patient continues to breathe during the procedure, maintaining adequate oxygen levels at the blood and organs and decreasing the potential for gastric content aspiration. The fastening of this apparatus is, therefore, essential to prevent the displacement of the tube that, besides impairing oxygenation, may cause injury to the airways.

In order to ensure fastening, materials suitable for this purpose should be used, such as laces or adhesives, the latter being preferred in cases of temporary surgical intubation. However, the current adhesive fasteners do not have a large area of attachment to the skin, increasing the risk that it will come loose from the patient's face and not isolating the oral cavity from the operative field, what may lead to contamination. Additionally, adhesive fasteners found in the prior art have a fastening method requiring the adhesive surface to be wrapped around the tube, which may be a difficult procedure for a person to do it alone and lead to waste of materials once the adhesive surface can stick to itself, making it difficult to use it, or stick to the professional performing the procedure's gloves.

It becomes clear, therefore, the need for a new apparatus that can be applied to the patient in a safe, fast and practical way and prevents the movement of the tube during the procedure, avoiding any accidental extubation.

### BACKGROUND OF THE INVENTION

The search in the state of the art for documents related to the fastening of endotracheal tubes disclosed MU8901057 4, entitled “Endotracheal Tube Fastener”, discloses a device to quickly and safely assist the fastening of endotracheal tubes. The device consists of a rigid body covering the entire mouth, said rigid body having a central hole for the passage of the endotracheal tube and provided with adhesive side strips fastening the device to the patient's face. The body is also provided with an adhesive tape that, when passed around the tube, keeps it attached.

Document CN203694317, entitled “Fastener Adhesive for Tracheal Catheter” discloses an adhesive tape comprising an adhesive layer and a protective layer thereof, which must be removed upon the application. The adhesive layer comprises a middle part, two fastening straps that attaches the tape to the upper part of the face, both connected to the left side and to the right side of the upper part of the middle part, and two fastening straps that attach the tape to the lower part of the face, also connected to the left side and the right side of the lower part of the middle part. The middle part also has a hole that can have different shapes, through which the endotracheal tube is inserted into the oral cavity and two middle tapes, one on each side that, at the time of intubation, are folded upwards and wrapped around the endotracheal tube, keeping it attached.

Finally, Document CN110392590 entitled “Endotracheal Tube Safety System and Method of Use Thereof” discloses a system having one or more basic articles which attaches the system to the patient's face, and one or more connecting articles, with one or more anchoring sections, configured to be connected to one or more basic articles. It also includes a capture section configured to hold the connecting article to an endotracheal tube. The embodiment of the system allows handling the tube without removing the base articles from the skin.

Other endotracheal tube fastening adhesives are also reported, such as those in an “I” shape having fastening straps allowing it to be attached to both the upper and lower part of the patient's mouth, but keeping the oral cavity uncovered during intubation.

Therefore, there are no inventions in the state of the art similar to either the fastening apparatus disclosed herein, or the use thereof, as proposed herein. Such invention, thus, meets the requirements for novelty and inventive step.

### BRIEF DESCRIPTION / SUMMARY OF THE INVENTION

One objective of the present invention is to disclose and teach an apparatus for fastening endotracheal tubes, specifically orotracheal tubes used in the advanced life support for patients undergoing surgical procedures and requiring general anesthesia.

The fastening apparatus of the present invention has its shape as a differential, assuring its best attachment to the patient's face, keeping the tube attached and stable, and reducing, thus, the risk of accidental extubation and hypoxemia. In addition, the apparatus can be made of disposable and low-cost material, allowing variations in size, which allows using it in newborns and adults, and replaces inappropriate means of fastening, such as plasters and micropore tapes.

Another advantage of the fastening apparatus of the present invention is to completely isolate the oral cavity from the surgical field, preventing the oral secretions from draining and reducing the risk of infection due to the entry of pathogenic microorganisms.

Another objective of the present invention is to teach and disclose the use of such fastening apparatus, keeping the endotracheal tube always at the same position and preventing accidental extubation during surgical procedures that require advanced life support, in addition to facilitating extubation at the end of the procedure.

Further features, advantages and objectives are discussed herein.

### BRIEF DESCRIPTION OF THE DRAWINGS

The subject matter of this invention will be totally clear in its technical aspects from the detailed description that will be made based on the figures listed below, to wit:

FIG. 1 is a plan view of the apparatus of the present invention without the endotracheal tube fastening flaps;

FIG. 2 is the plan view of FIG. 1 with the endotracheal tube fastening flaps;

FIG. 3 shows the actual representation of the fastening apparatus of the present invention;

FIG. 4 illustrates the steps for applying the fastening apparatus of the present invention to a patient;

FIG. 5 illustrates the steps for withdrawing the fastening apparatus of the present invention;

FIG. 6 shows the endotracheal tube fitted to the apparatus of the present invention;

FIG. 7 shows how the fastening apparatus of the present invention is attached to the endotracheal tube. In this figure, it can also be seen the protective layers of the adhesive layer and the flaps that are attached to the tube withdrawn from the apparatus;

FIG. 8 shows the fastening flaps the endotracheal tube folded upwards, forming a perpendicular angle with the adhesive layer and, consequently, with the protective layer; and

FIG. 9 shows the protective layer of the adhesive layer, divided into two parts, and each of the fastening flaps of the endotracheal tube.

### DETAILED DESCRIPTION OF THE INVENTION

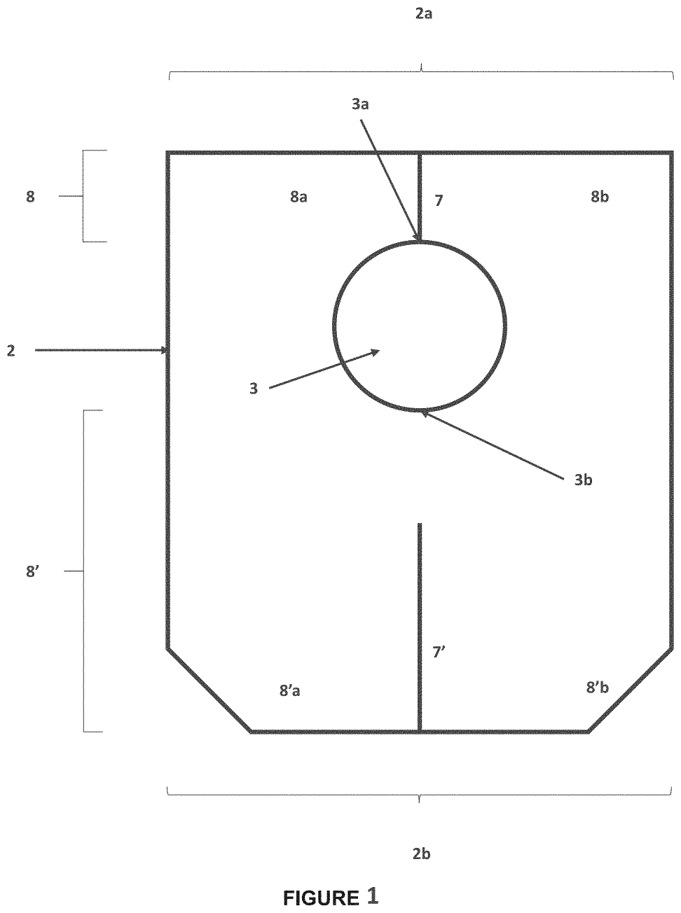

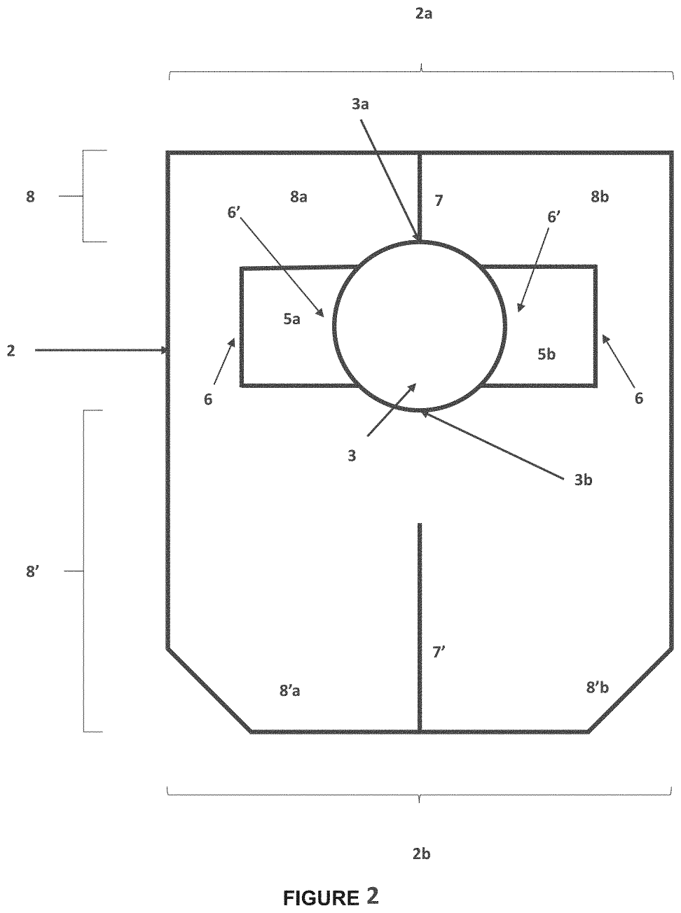

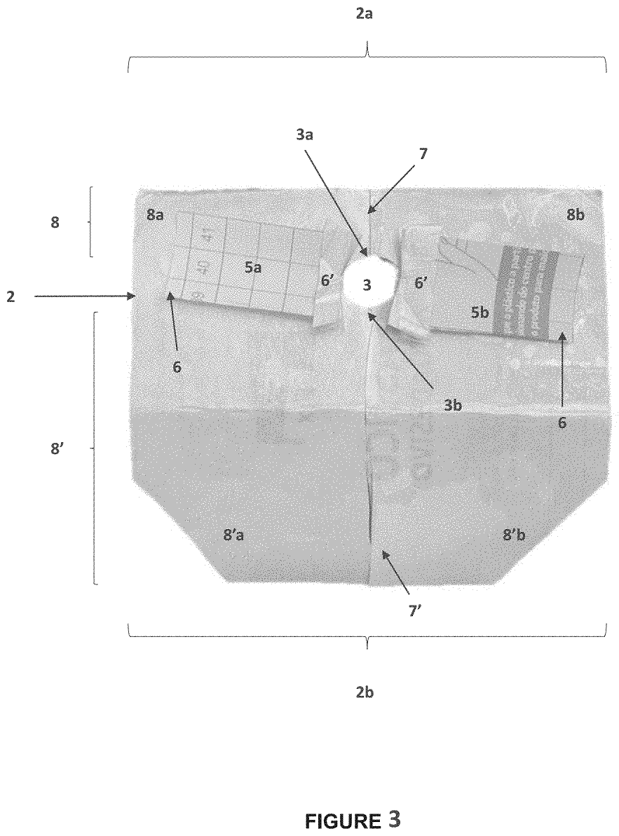

In accordance with the objectives presented through the brief description, the present patent application shows an ENDOTRACHEAL TUBE FASTENING APPARATUS (1), as showed in the plan views of FIG. 1, FIG. 2 and FIG. 3, comprising an adhesive layer (2) and a hole (3) for the passage of an endotracheal tube (4), wherein the adhesive layer (2) is divided into two portions, one smaller upper (8) and one larger lower (8′) part and has enough dimension to allow its fastening, in a simultaneous manner, on the upper part of the lip and on the mental region of a patient;

wherein at the end (2a), facing the upper lip, of the apparatus (1) there is a cutout (7) extending to the hole (3), and an opening (3a) is formed in it, also facing the upper lip, and divides the smaller upper (8) portion of the apparatus (1) into two upper attachments (8a, 8b);

wherein there is also a cutout (7′) in the larger lower (8′) portion of the adhesive layer (2), starting at the end (2b), facing the mental region of the apparatus (1) and is large enough to enable the adhesion of layer (2) on the skin and keep intact the contour (3b) also facing the mental region of the hole (3), so that the cutout (7′) also divides the larger lower portion (8′) into two lower attachments (8′a, 8′b);

said hole (3) has, on its sides, two flaps (5a, 5b), one on each side, the said flaps (5a, 5b) having each a first free end (6) and a second end (6′) adjacent to the hole (3), attached to the adhesive layer (2); the said flaps (5a, 5b) can be folded upwards, forming a channel (5c) and are also formed by an adhesive layer (5).

Referring also to FIGS. 1, 2, and 3, in an embodiment of the apparatus of the present invention, the adhesive layer (2) has a beveled rectangular shape, with the bevels of the layer (2) located at the end (2b) of the larger lower portion (8′), facing the region to be fixed on the mental region and, therefore, opposite to the end (2a) of the apparatus (1). The adhesive layer (2) also has a hole (3) applied in such a way that, when applying the apparatus (1) to a patient, the hole (3) is positioned at the center of the mouth.

In another embodiment of the apparatus (1), as it can also be seen in FIGS. 1, 2, and 3, the cutout (7, 7′) is positioned in the middle region of the upper (2a) and lower (2b) extremities of the apparatus (1) and towards the center of the hole (3).

The size of the adhesive layer (2), as mentioned, must be such that it can be simultaneously attached above the patient's upper lip, and on his/her mental region (chin), considering previously that several sizes must be predicted taking into account the possibility of intubation of premature babies or adults, having the most diverse body and facial structures. In this way, the apparatus (1) of the present invention can be made in different sizes in order to allow the application in a great diversity of patients needing intubation for any surgical procedure that requires advanced life support.

The size of the hole (3), in the same way as the size of the adhesive layer (2), must be considered according to the different diameters of existing endotracheal tubes, which must be chosen taking into account the patient's height, physiological condition and age. In this way, the apparatus (1) can be made with endotracheal tube passage holes of different diameters, also allowing its use in surgeries of premature babies, newborns, children and adults with the most diverse body structures.

Thus, the same size of the layer (2) can be made with different sizes of hole (3), made to house the diameters of endotracheal tubes available in the market.

Due to its innovative shape, and different from fastener apparatuses comprising an adhesive layer of the state of the art, the method of application of the apparatus (1) of the present invention is different and equally advantageous, enabling the endotracheal tube to be securely and easily fasten, in addition to ensuring its withdrawn without causing injuries patient's extubation.

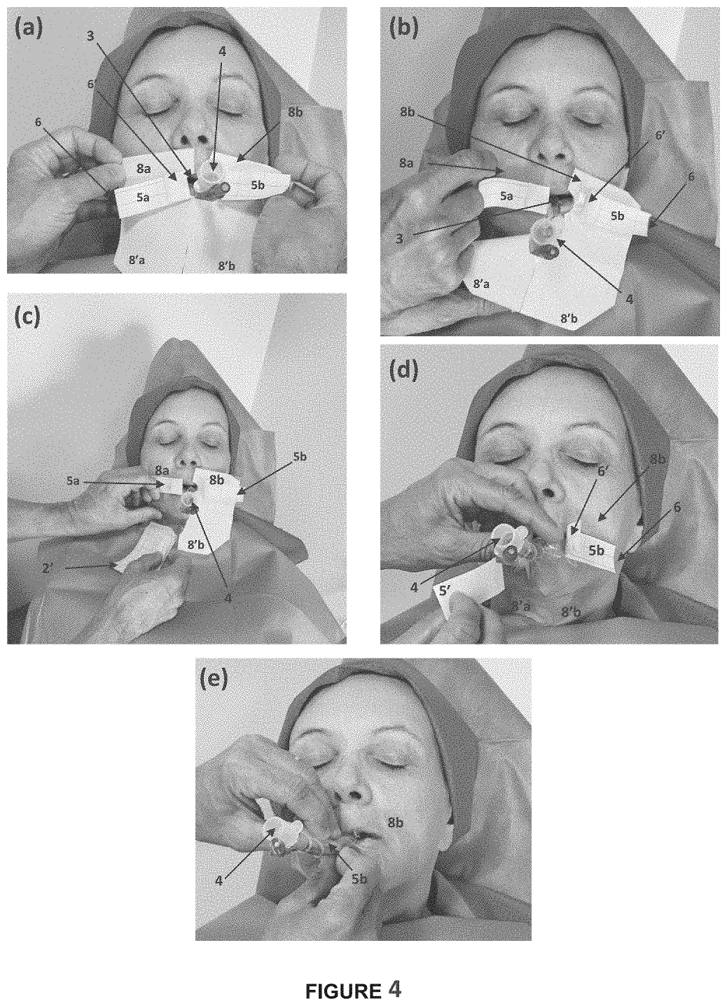

The application comprises the following steps, which are illustrated in FIG. 4:

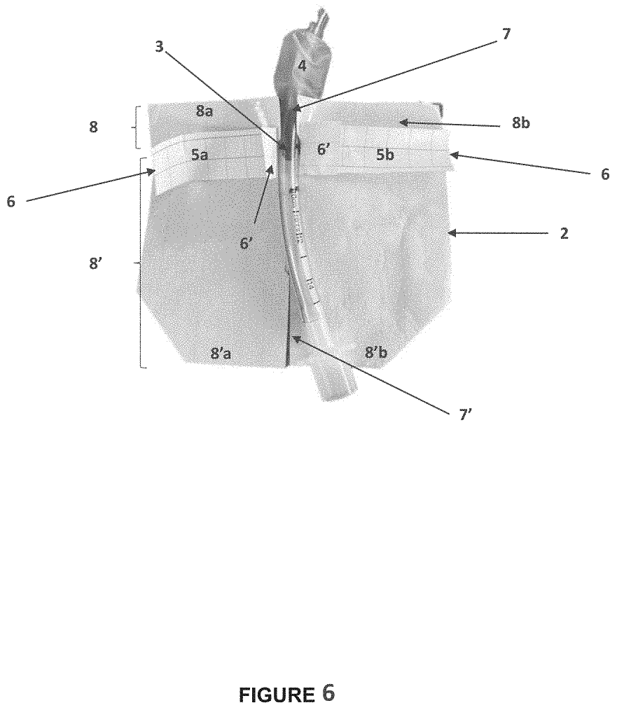

to pass the endotracheal tube (4) through the hole (3) of the apparatus (1), as shown in FIG. 4(a). FIG. 6 illustrates an endotracheal tube (4) wrapped by the apparatus (1) without being applied to a patient's face for a better understanding of how the tube (4) passes through the hole (3). Still with respect to FIG. 6, it can be seen how the opening (3a), not shown in the figure, but shown in FIG. 3, facing the lower lip, of the hole (3) opens, this opening being formed by the cutout (7) for fitting the tube while the contour (3b), below the tube and also not referenced in this figure, keeps intact and supports the tube (4);

to position the two upper attachments (8a, 8b) of the apparatus (1) towards the sides of the nose and above the upper lip of the patient, as shown in FIGS. 4(a) and 4(b);

to remove partially the second layer (2′) from each upper attachments (8a, 8b) and attach them to the skin, as can be seen in FIG. 4(b);

after attaching the smaller upper (8) portion of the apparatus (1) to the skin, to remove the remainder of the second layer (2′) and gradually attach the adhesive layer (2) on both sides of the mouth and on the mental region, as can be seen in FIG. 4(c); and

after the adhesive layer (2) of the apparatus (1) is completely attached to the patient's skin, to remove the protective layer (5′) from the flaps (5a, 5b) and attach them to the endotracheal tube (4), as can be seen in FIG. 4(d). In FIG. 4(d), it can also be seen the lower attachment (8′b) already attached to the patient's skin. FIG. 4(e) shows the apparatus (1) fully installed, with the two flaps (5a, 5b) fixed to the tube (4) and the upper attachment (8b) completely fastened to the skin.

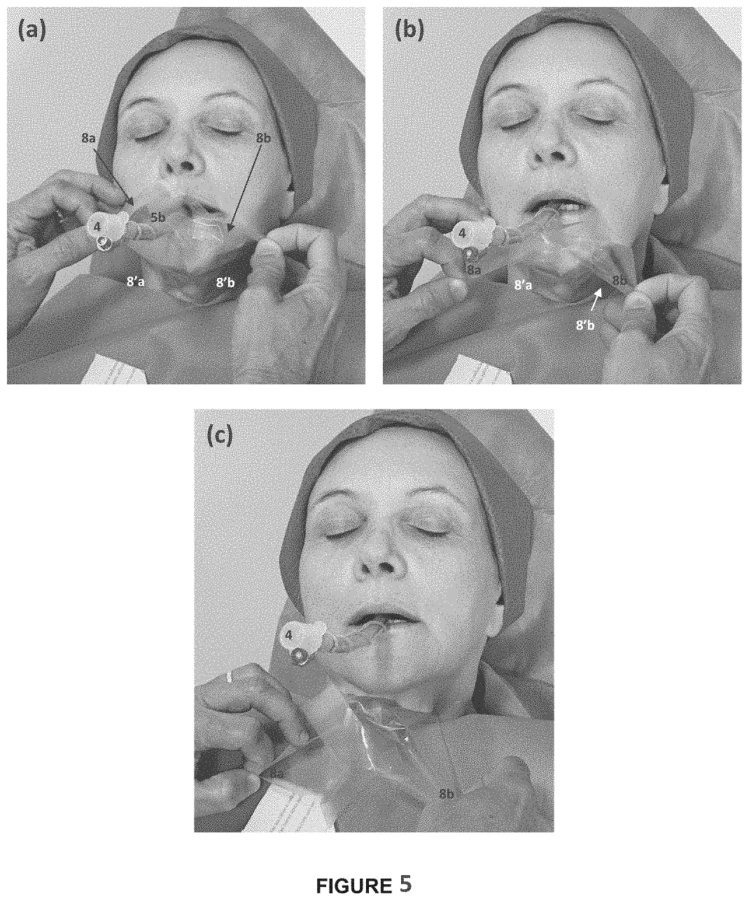

The withdraw of the endotracheal tube, illustrated in FIG. 5, is also facilitated by the shape of the apparatus (1), made by first detaching the flaps (5a, 5b) from the tube (4), undoing, thus, the channel (5c); then, the two upper attachments (8a, 8b) of the apparatus (1) while the two lower attachments (8′a, 8′b) still keep attached to the patient's mental region, ensuring the stability of the tube during the handling, since it is not necessary to handle it to withdraw the apparatus (1).

According to the aforementioned and in line with an embodiment of the apparatus (1) of the present invention, the apparatus (1) when applied to the face of a patient submitted to a surgery that requires advances life support, completely covers the oral cavity, isolating it and reducing the risk of contamination during the procedure, with the hole (3) positioned at the center of the mouth.

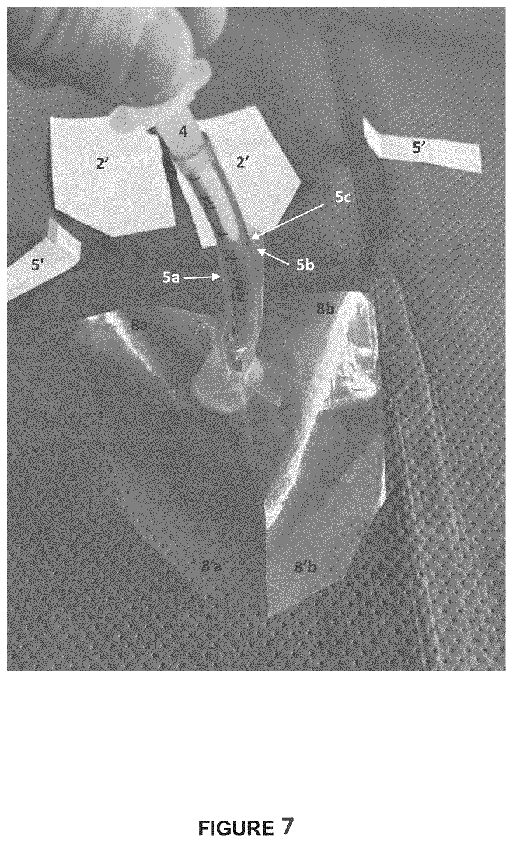

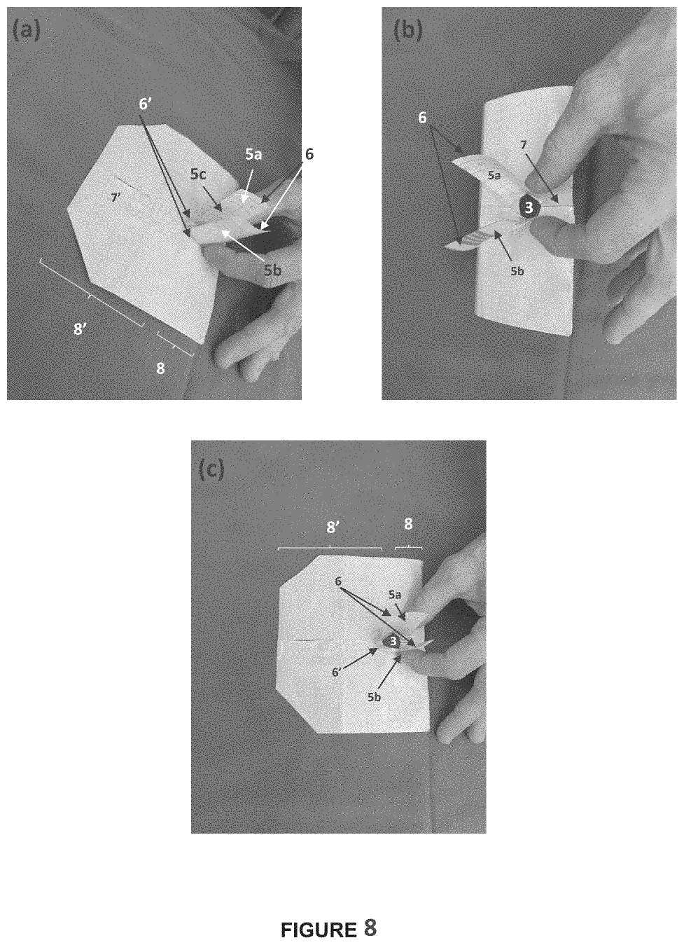

In another embodiment of the apparatus (1), as shown in FIG. 7, the flaps (5a, 5b) are attached to the endotracheal tube (4). For the flaps (5a, 5b) to be attached to the tube (4), the second ends (6′) of the flaps (5a, 5b) are folded upwards, forming an adhesive channel (5c), causing said flaps (5a, 5b), and consequently the channel (5c), to be perpendicular to the adhesive layer (2), as shown in FIG. 8(a, b and c).

In another embodiment of the apparatus (1), also illustrated in FIG. 7, the flaps (5a, 5b) are attached to the endotracheal tube (4) in the longitudinal direction of said tube (4) and are parallel to each other.

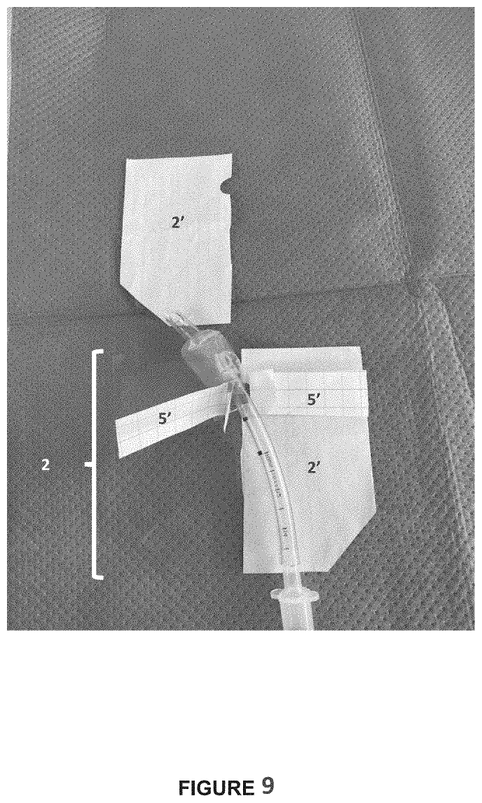

In a last but one embodiment of the apparatus (1), the adhesive layer (2, 5) has a second protection layer (2′, 5′) in the same shape and size as the protected adhesive layer (2, 5). In a last embodiment of the apparatus (1), the second protection layer (2′) is divided into two parts, in the longitudinal direction, to facilitate the handling of the apparatus (1) during application, as can be seen in FIG. 9.

The present patent application also shows the use of an apparatus (1), as defined herein, for fastening an endotracheal tube (4) during surgical procedures that make it necessary to provide the patient with advanced life support.

It should be understood the present specification does not limit the application to the details described herein and the invention is capable of other embodiments and to be practiced or performed in a variety of ways within the scope of the claims. Although specific terms have been used, these terms should be construed in a generic and descriptive sense, and not for the purpose of limitation.

What is claimed is:

1. An endotracheal tube fastening apparatus comprising:
an adhesive layer and a hole for passing an endotracheal tube characterized in that the adhesive layer is divided into two portions, being one smaller upper and one larger lower portion and of sufficient size to allow its simultaneously fastening on the upper part of the lip and in a mental region of a patient, wherein:
at the end facing the upper lip of the apparatus there is a cutout extending to the hole, and opening is formed in it, also facing the upper lip and dividing the smaller upper portion of the apparatus into two upper attachments;
at the larger lower portion of the adhesive layer, there is also a cutout starting at the end, facing the mental region of the apparatus and being of sufficient size to allow the adhesion of the layer on the skin and to keep intact the contour, also facing the mental region of the hole; said cutout also dividing the larger lower portion into two lower attachments;
on its sides, the hole has two flaps, one on each side, the said flaps having each a first free end and a second end adjacent to the hole, attached to the adhesive layer, wherein:
the said flaps are also formed by an adhesive layer; and
the said flaps can be folded upwards, forming a channel.

2. The Apparatus, according to claim 1, characterized in that the adhesive layer has a rectangular beveled shape.

3. The Apparatus, according to claim 2, characterized in that the bevels of the adhesive layer are located at the end of the larger lower portion, facing the mental region and opposite to the end of the apparatus.

4. The Apparatus, according to claim 1, characterized in that the hole is made in the adhesive layer in such a way it is positioned at the center of the mouth when the apparatus is applied.

5. The Apparatus, according to claim 1, characterized in that the cutout is positioned in the middle region of the upper and lower ends of the apparatus and towards the center of the hole.

6. The Apparatus, according to claim 1, characterized in that the adhesive layer, when applied, fully covers the oral cavity.

7. The Apparatus, according to claim 1, characterized in that the flaps are attached to the endotracheal tube.

8. The Apparatus, according to claim 7, characterized in that, to fast the endotracheal tube, the second ends of the flaps are folded upwards, forming an adhesive channel for the tube, causing said flaps, and consequently the channel, to be perpendicular to the adhesive layer.

9. The Apparatus, according to claim 7, characterized in that the flaps are attached to the endotracheal tube in the longitudinal direction of said tube and parallel to each other.

10. The Apparatus, according to claim 1, characterized in that each adhesive layer has a second protective layer of same shape and size as the adhesive layer.

11. The Apparatus, according to claim 10, characterized in that the second protective layer is divided into two parts in the longitudinal direction.

12. Use Of An Apparatus, as defined in claim 1, characterized in that the said use is for fastening an endotracheal tube during surgical procedures.
